(19) United States
(12) Patent Application Publication
(10) Pub. No.: US 2016/0346091 A1
(43) Pub. Date: Dec. 1, 2016

(54) A METHOD FOR MANUFACTURING A CUSTOMIZED IMPLANT

(71) Applicant: UNIVERSITI MALAYA, W.P. Kuala Lumpur (MY)

(72) Inventors: Zainal Ariff Bin Abdul Rahman, KUALA LUMPUR (MY); Vickneswaran A/L Mathaneswaran, KUALA LUMPUR (MY); Yuwaraj Kumar A/L Balakrishnan, KUALA LUMPUR (MY); Su Tung Tan, KUALA LUMPUR (MY); Alwin Kumar Rathinam, KUALA LUMPUR (MY)

(21) Appl. No.: 15/100,325

(22) PCT Filed: Apr. 21, 2014
(86) PCT No.: PCT/MY14/00065

§ 371 Date: May 30, 2016

(30) Foreign Application Priority Data
Apr. 30, 2013 (MY) PI 2013700701

(51) Int. Cl. A61F 2/30; B29C 67/00; A61F 2/28

(52) U.S. Cl. CPC A61F 2/30942; A61F 2/2875; B29C 67/0051; A61F 2002/30948; A61F 2002/3097; A61F 2002/30985; A61F 2002/30962; A61F 2310/00023; B33Y 30/00

(57) ABSTRACT

The present invention relates to a method for manufacturing a customized implant comprising the steps of obtaining a plurality of medical images of a bone region with a defect area; converting the medical images into a three-dimensional data; designing a mould customized for the defect area based on the three-dimensional data to produce a customized mould; and fabricating a customized implant from a biocompatible plate using the customized mould via an additive manufacturing technique.

### FIELD OF INVENTION

This invention relates to a method for producing a customized implant. In more particular, this invention relates to a method for producing a customized implant, especially a cranioplasty implant, using an additive technology.

### BACKGROUND OF THE INVENTION

Defects on human skull or bone may be caused by injuries, diseases, surgical interventions or congenital abnormalities. Fortunately, the defects are mostly repairable or reconfigurable by surgical implants. Therefore, the surgical implants must consist high similarity to the shape or contour of the patient's bone structure for a desirable appearance.

While several processes such as milling, drilling or turning are commonly used to produce such implants, these processes are relatively wasteful as materials from the work piece are cut off to form the desired implants. Even if the implants are made from machining or casting, only certain materials can be used. As surgical implants are substantially inserted into the patient's body, the materials must be essentially biocompatible and any infection must be prevented.

There are some patent technologies over the prior arts relating to methods to produce three-dimensional (3D) models. Of interest is a U.S. Patent No. US 2005/0133955(A1), disclosing a method for designing and producing a custom-fit prosthesis. A two-part mould is manufactured based on medical image data. However, the mould is meant to be used for injection moulding.

Another U.S. Patent No. US 2006094951(A1) discloses a method to produce an implant for a patient prior to operation. The method comprises generating data that represent an area that will receive the implant, designing the implant and fabricating the implant. This invention focuses on the fabrication of the implant directly from a rapid prototyping technology but not from moulds or 3D data of medical images.

Similarly, another U.S. Patent No. US 2011144752 (A1) discloses a method for manufacturing customized implant by using a computer-based imaging and rapid prototyping-based manufacturing technique. The customized implant is formed using a solid free-form fabrication method comprising sequential layers of polyether ketone powder. However, this prior art focuses on direct manufacturing of implants but not on manufacturing of moulds for implants. Thus, this invention is not capable of producing implants press-moulded from biocompatible plate.

Mesh plates are normally fabricated by machining thin plates and forming multiple millimeter sized perforations on the plates. Due to the inability of a machine to simultaneously fabricate thin meshed plates together with organically curved implants directly from an additive manufacturing technique, a need therefore raises to produce an implant formed by a mould with desired shape or contour using press moulding technique of a commercially available mesh plate.

### SUMMARY OF INVENTION

One of the objects of the present invention is to construct a customized implant fabricated from a mould produced according to medical images.

Another object of the present invention is to produce a customized implant fabricated from a biocompatible plate through press moulding. The customized implant is produced in a relatively fast and cost effective method.

It is yet another object of the present invention to provide a customized implant that fits accurately to a defect area of a bone structure. Designs or modifications are carried out before fabrication to reduce surgical procedures, time of surgery and increase accuracy of implant to patient's defect area.

At least one of the proceeding objects is met, in whole or in part, by the present invention, in which the preferred embodiment of the present invention describes a method for manufacturing a customized implant comprising the steps of obtaining a plurality of medical images of a bone region with a defect area; converting the medical images into a 3D data; designing a mould customized for the defect area based on the 3D data to produce a customized mould; and fabricating a customized implant from a biocompatible plate using the customized mould via an additive manufacturing technique.

One of the preferred embodiments of the present invention discloses that, the customized implant is a cranioplasty plate.

In accordance with a preferred embodiment of the present invention, the medical images are X-ray images, computed tomography images, magnetic resonance images, ultrasound images, positron emission tomography images or single-photon emission computed tomography images.

Preferably, the medical images are converted into the 3D data using a Marching cube algorithm, a Delaunay's triangulation algorithm or a combination thereof.

Another preferred embodiment of the present invention discloses that the biocompatible plate is a titanium mesh plate or an acrylic plate.

Still another preferred embodiment of the present invention discloses that the additive manufacturing technique includes rapid layered manufacturing, direct digital manufacturing, laser processing, electron beam melting, aerosol jetting, inkjet printing, semi-solid free-form fabrication or a combination of any two or more thereof.

The present preferred embodiments of the invention consists of novel features and a combination of parts hereinafter fully described and illustrated in the accompanying drawings and particularly pointed out in the appended claims; it being understood that various changes in the details may be effected by those skilled in the arts but without departing from the scope of the invention or sacrificing any of the advantages of the present invention.

### DETAILED DESCRIPTION OF THE INVENTION

Hereinafter, the invention shall be described according to the preferred embodiments of the present invention and by referring to the accompanying description and drawings. However, it is to be understood that limiting the description to the preferred embodiments of the invention and to the drawings is merely to facilitate discussion of the present invention and it is envisioned that those skilled in the art may devise various modifications without departing from the scope of the appended claim.

This invention relates to a method for producing a customized implant. In more particular, this invention relates to a method for producing a customized implant, especially a cranioplasty implant, using an additive technology.

The present invention discloses a method for manufacturing a customized implant comprising the steps of obtaining a plurality of medical images of a bone region with a defect area; converting the medical images into a 3D data; designing a mould customized for the defect area based on the 3D data to produce a customized mould; and fabricating a customized implant from a biocompatible plate using the customized mould via an additive manufacturing technique.

According to one of the preferred embodiments of the present invention, a plurality of medical images can be obtained from a patient or any biological organism. The medical images show a bone region with defect areas that require replacement or repair by an implant. In accordance with the most preferred embodiment, the implant fabricated is a cranioplasty plate for use in the skull. Accordingly, each medical image is preferred to be segmented to obtain images with non-other-than the bone region, eliminating any unwanted void regions. The plurality of images are rendered together to produce a 3D image showing the bone region. It is to be understood that the medical images are images in the transverse, coronal or sagittal planes of a patient or biological organism and the planes depend on a diagnostic task. The medical images may be any images that are capable of capturing bone regions of a patient or biological organism. The medical images can be otherwise referred to as medical scan images. They can be X-ray images, computed tomography images, magnetic resonance images or any other medical images.

Accordingly, the medical image has a plurality of regions having different grey level values. The regions are shown by multiple volumetric pixels and each pixel correspondences to a grey level value. The grey level values range from 0-255 for images with 8-bits per pixel. The medical images generally has a void region having the darkest shade, represented with a grey level value of 0, while the bone regions have lighter shades than the void region with grey level values in a range of 1 to 255 for a similar 8-bit per pixel image. Preferably, through segmenting, the void region is eliminated and the bone region is selected and subsequently converted to a 3D data. Upon segmentation, any noise, artifacts or undesired regions are preferred to be eliminated or reduced.

According to another embodiment of the present invention, the medical images shows that each pixel has an intensity of grey shade, where the weakest intensity is black, the strongest intensity is white and many shades of grey in between. For medical images with colour scales, the images are preferred to be converted to greyscale images as vectorization of coloured images produces poor results. The medical images are preferred to be analyzed in a computing device and the intensity of the grey shades are computed through the grey level values that can be stored in binary or quantized forms. The values are converted to vector data by a mathematical equation, preferably a linear equation. The vector data is preferred to be in forms of arcs and lines that are geometrically and mathematically associated. The vector data is stored as a series of pixel pairs, preferably in a polygon (PLY) file format as it is simple, fast in saving and loading as well as easy to be implemented for a wide range of computer programmes.

A particular embodiment of the present invention discloses that the step of converting the segmented medical images into the 3D data is by using a Marching cube algorithm, Delaunay's triangulation algorithm or a combination thereof. Marching cube algorithm, Delaunay's triangulation algorithm or the combination thereof are preferred to be used due to its isotropic ability to expand pixels of the vector data in a single direction. The pixels in the medical images are interpolated to form connecting series of pixel pairs. Eventually, printing of the 3D data fabricates a 3D customized mould with a continuous and smooth surface. The 3D data is preferred to be initially designed or modified in the computing device to generate the mould having a mould cavity that is able to mould out implants which totally match and fit the defect areas of the bone region. The design or modification can be carried out according to patient's need for better appearance.

According to yet another embodiment of the present invention, the 3D data is subjected to an additive manufacturing technique where layers of material are added upon one another to form the desired customized mould. The rapid additive manufacturing technique includes layered manufacturing, direct digital manufacturing, laser processing, electron beam melting, aerosol jetting or semi-solid free-form fabrication. The technique rapidly and sequentially built up many thin layers upon one another to produce the customized mould.

By way of manufacturing the mould, several advantages can be obtained. Preferably, the mould designed and produced by the present invention is a negative mould, in which the biocompatible plate can be directly press-moulded thereon. The customized mould is able to mould out the customized implant using a preferred implant material, which is a biocompatible plate such as thin titanium mesh or acrylic plate. As embodied in one of the preferred embodiments of the present invention, titanium mesh plate is more preferred to be used as the implant material as it is able to resist corrosion, is biocompatible and having an innate ability to join with bone. It is also having high strength yet light weight properties. The perforated structure of the mesh plate enhances better blood miscibility, thus providing a long term acceptance of tissues. The customized implant that resembles actual bone region of the patient or biological organism is used for covering or replacing the defect region of the bone region.

Cold press moulding is a preferred technique to produce the customized implant. The implant material such as titanium mesh plate is preferably pressed onto the mould cavity in room temperature or without heating. The titanium mesh plate is preferred to be gradually moulded by cold press moulding so as to maintain chemical and physical properties of the titanium mesh plate.

The customized implant is meant to be placed on the defect area of the bone region where repairing or re-shaping is needed. The defect area may be a missing bone, a crack or merely undesired shape. The customized implant produced from the pressing method is preferred to be seamlessly and smoothly compatible to the bone region.

Surgical procedure can be shortened as the customized implant fits well to the patient's defect area and modification during surgery is therefore not needed. Patient's surgery risk will be prevented or reduced as well.

While the invention has been disclosed in connection with the preferred embodiments shown and described in detail, various modifications and improvements thereon will become readily apparent to those skilled in the art. Accordingly, the spirit and scope of the present invention is not to be limited by the foregoing examples, but is to be understood in the broadest sense allowable by law.

### EXAMPLE

An example is provided below to illustrate different aspects and embodiments of the present invention. The example is not intended in any way to limit the disclosed invention, which is limited only by the claims.

The pixels in the medical images are preferred to be connected to one another by the linear equation to form a vector data. The equation forms a straight line in the plane between two pixels or points. The linear equation as described in accordance to an embodiment of the present invention is as follows:

f(x)=mx+c

where m is slope or gradient of the line, x is a point at which the line crosses the x-axis and c is a point at which the line crosses the y-axis, otherwise known as the y-intercept.

Each points are joined to one another by the linear equation to form the vector data. The vector data are subsequently converted to 3D data by Marching cube and Delauney's algorithm.

1.-8. (canceled)

9. A method for manufacturing a customized implant, comprising the steps of:
obtaining a plurality of medical images of a bone region with a defect area;
converting the medical images into three-dimensional data;
designing a mould customized for the defect area based on the three-dimensional data to produce a customized mould via an additive manufacturing technique; and
fabricating a customized implant from a biocompatible plate using the customized mould.

10. The method according to claim 9, wherein the customized implant is a cranioplasty plate.

11. The method according to claim 9, wherein the medical images are X-ray images, computed tomography images, magnetic resonance images, ultrasound images, positron emission tomography images or single-photon emission computed tomography images.

12. The method according to claim 9, wherein the medical images are converted into the three-dimensional data using a Marching cube algorithm, a Delaunay's triangulation algorithm or a combination thereof.

13. The method according to claim 9, wherein the biocompatible plate is a titanium mesh plate or an acrylic plate.

14. The method according to claim 9, wherein the additive manufacturing technique includes rapid layered manufacturing, direct digital manufacturing, laser processing, electron beam melting, aerosol jetting, inkjet printing, semi-solid free-form fabrication or a combination of any two or more thereof.
